(19) United States
(12) Patent Application Publication
(10) Pub. No.: US 2021/0255084 A1
(43) Pub. Date: Aug. 19, 2021

(54) METHOD FOR DETERMINING A QUANTITY OF GAS ADSORBED IN A POROUS MEDIUM

(71) Applicant: IFP Energies nouvelles, Rueil-Malmaison (FR)

(72) Inventors: Guillaume BERTHE, RUEIL-MALMAISON (FR); Frederic MARTIN, RUEIL-MALMAISON (FR); Farah AL SAHYOUNI, RUEIL-MALMAISON (FR)

(21) Appl. No.: 17/268,855

(22) PCT Filed: Jul. 9, 2019
(86) PCT No.: PCT/EP2019/068350

§ 371 Date: Feb. 16, 2021

(30) Foreign Application Priority Data
Aug. 16, 2018 (FR) 1857508

(51) Int. Cl. G01N 15/08

(52) U.S. Cl. CPC G01N 15/0826; G01N 15/088

(57) ABSTRACT

The invention relates to a method of determining at least one quantity relative to the adsorption of at least one adsorbable gas in a sample of a porous medium, wherein the following steps are carried out: (i) determining a Darcy velocity by injecting an inert gas for a given gradient and by measuring the inert gas flow rate downstream from the sample, (ii) determining an adsorbable gas breakthrough velocity by injecting the adsorbable gas for the same gradient and by measuring the adsorbable gas quantity downstream from the sample as a function of time, and (iii) determining a kinematic porosity using the ratio of the Darcy velocity to the adsorbable gas breakthrough velocity.

### FIELD OF THE INVENTION

The present invention relates to the field of petrophysical characterization of a porous medium, notably the characterization of the adsorption capacity of a porous medium in which at least one gas is present.

The porous medium according to the invention can be a rock sample from an underground formation: in this case, the method according to the invention is applicable in the field of exploration and exploitation of petroleum reservoirs or of geological storage sites for gases such as CO₂ or methane. The porous medium according to the invention may however also concern a catalyst, a concrete piece or filter membranes.

The present invention is described hereafter by way of non-limitative example within the context of petrophysical characterization of a rock from an underground formation. In particular, the present invention can be advantageously applied in the case of a low-permeability rock (tight rock). The present invention finds a particular application in the petrophysical characterization of rocks containing gases commonly referred to as shale gas or source rock gas.

Shale gas is a gas mainly consisting of methane contained in clayey rocks with a high organic matter content. These clays (actually often a mixture of clays, silt or carbonates) have been buried deep enough for the organic matter to be transformed into gas. A large part of this gas remains trapped in the clays because they are almost impermeable and very adsorptive.

The gas production potential of such a rock therefore depends in particular on the gas adsorption capacity of this rock. The adsorption capacity corresponds to the adsorptive power of a porous medium.

### BACKGROUND OF THE INVENTION

The following documents are mentioned in the description:

F. M. Nelsen and F. T. Eggertsen, 1958, Determination of Surface Area. Adsorption Measurements by Continuous Flow Method, Analytical Chemistry, 30 (8), 1387-1390, DOI: 10.1021/ac60140a029.

J. H. Atkins, 1964, Rapid and Precise Method for Determining Surface Areas, Analytical Chemistry, 36 (3), 579-58, DOI: 10.1021/ac60209a007.

S. Karp, S. Lowell, and A. Mustacciuolo, 1972, Continuous Flow Measurement of Desorption Isotherms, ANALYTICAL CHEMISTRY, VOL. 44, NO. 14, DECEMBER 1972, 2395-2397.

In general terms, adsorption is a well-known physical phenomenon that is however very difficult to quantify experimentally.

Conventionally, plotting a physical adsorption isotherm requires measuring the quantity adsorbed as a function of the relative equilibrium pressure of the adsorbable gas. The three most commonly used methods of the prior art are:
the volumetric or manometric adsorption method: previously, this method used mercury-filled burettes for varying the volume occupied by the gas. In current devices, the volume occupied by the gas remains constant and measurement of the adsorbed quantity is based on the measurement of the adsorbable gas pressure, the temperature being kept constant. In this technique, measurement of the gas pressure allows to know both the equilibrium pressure and the adsorbed quantity. The accuracy with which the adsorbed quantity is measured depends not only on the accuracy with which the pressure of and the volume available to the gas phase are measured, but also on the equation of state used to describe the adsorbable gas,
the gravimetric adsorption method: in the case of this method, the adsorbent is directly put in a balance designed for adsorption; the mass of the adsorbent can then be monitored permanently during the adsorption process. It can be noted that it is also necessary to measure the pressure of the gas phase at equilibrium with the adsorbed phase as well,
desorption in a carrier gas flow: in the case of this method, notably described in the documents (Nelsen and Eggertsen, 1958; Atkins, 1964; Karp et al., 1972), the quantity of adsorbed dinitrogen is measured during a sudden desorption, with entrainment by a carrier gas and detection using a TCD detector. Due to the equipment and to the procedure implemented, this method is often mistakenly referred to as “chromatographic”, although it is not based on the principle of chromatography. Indeed, within the context of this method, the sample is first brought, at 77° K, at equilibrium with a flow of dinitrogen and helium whose partial dinitrogen pressure thus becomes the adsorption pressure. Helium is a very good heat conductor and it promotes rapid equilibrium (a few minutes are often sufficient). However, to benefit from a good quality TCD signal, it is during a very fast desorption (obtained by heating to ambient temperature) that the additional quantity of dinitrogen then carried along by the gas flow is measured. The large thermal conductivity difference between helium and dinitrogen ensures optimum performance of the TCD detector (using a heated filament, more or less cooled by the gas flow). The surface area of the peak recorded during desorption is proportional to the desorbed quantity. The proportionality constant is determined by calibration, by injecting a known quantity of nitrogen into the pure helium.

Patent FR-2,999,716 that describes a method allowing characterization of the migration by adsorption of a gas in a porous medium is also known. A steady-state test is first carried out on a rock sample using an inert gas as the tracer gas, during which a first tracer gas flow is measured, from which a migration by advection is characterized within the sample. Then, a steady-state test is carried out on this sample using methane as the tracer gas, during which a second tracer gas flow is measured. A third flow of methane passing through the sample by advection is then estimated by means of the first test. Then, the flow of methane passing through the sample by adsorption is deduced from the third flow and the second flow. Finally, the migration by adsorption is characterized by the methane flow passing through the sample by adsorption. However, this method does not allow to obtain a direct teaching on the gas adsorption capacity of the sample. Indeed, this method provides a teaching relative to the migration by adsorption, but not to the adsorbed gas quantity. Besides, the device used is relatively complex since it requires a dual outlet downstream from the experimental setup (the downstream face of the sample is swept by a carrier gas).

The present invention represents an alternative to known methods of the prior art for determining a quantity of gas adsorbed in a porous medium. In particular, the method according to the invention allows, from conventional experimental measurements, fast and easy to implement, to determine in a reliable and robust manner a quantity, relative to the adsorption of an adsorbable gas in a sample of a porous medium. Indeed, the present invention allows the method to be carried out by means of experimental setups conventionally used for measuring low-permeability porous media. Besides, compared with patent FR-2,999,716, the present invention requires no dual outlet downstream from the experimental setup.

### SUMMARY OF THE INVENTION

The present invention relates to a method of determining at least one quantity relative to the adsorption of at least one adsorbable gas in a sample of a porous medium. The method according to the invention comprises at least the following steps:
a) applying a pressure gradient between upstream and downstream of said sample and injecting an inert gas upstream from said sample subjected to said pressure gradient; measuring at least one flow rate of said inert gas downstream from said sample, and determining a Darcy velocity from said flow rate of said measured inert gas,
b) for said pressure gradient applied between upstream and downstream of said sample, said sample being saturated with said inert gas, injecting an adsorbable gas upstream from said sample at a first time t, said adsorbable gas having a concentration C_{g}; downstream from said sample and for a plurality of times later than said first time, measuring a quantity of said adsorbable gas that has passed through said sample; determining a breakthrough velocity for said adsorbable gas from the time t′ of a maximum of the curve representative of the time-dependent evolution of said measured adsorbable gas quantity for said plurality of times,
c) determining a kinematic porosity as a function of said pressure gradient applied to said sample and of said concentration in said adsorbable gas from the ratio of said Darcy velocity to said breakthrough velocity of said adsorbable gas, and determining, for said pressure gradient applied to said sample and for said concentration in said adsorbable gas, a quantity relative to said adsorption of said adsorbable gas in said sample, from said kinematic porosity.

Preferably, in step B, the volume of said injected adsorbable gas can be less than the volume of the pores of said sample.

Advantageously, said breakthrough velocity Vₜ of said adsorbable gas can be determined with a formula of the type: Vₜ(ΔP,C_{g})=Δt/L, where Δt=t′-t, L is the length of said sample, ΔP is said pressure gradient and C_{g} is said concentration in said adsorbable gas.

According to an implementation of the invention, said quantity relative to the adsorption can be a volume of gas adsorbed in said sample and/or a mass of gas adsorbed in said sample.

According to an embodiment of the invention, said adsorbed gas volume in said sample can be determined with a formula of the type:

Vg(Δp,Cg)=V·(Φ−ωc(Δp,Cg)), in m³,

where V is the volume of said sample; Φ is the total porosity of said sample and ωc(Δp,Cg) is said kinematic porosity determined for said pressure gradient ΔP and said adsorbable gas concentration C_{g}.

According to another embodiment of the invention, said adsorbed gas mass mg in said sample can be determined with a formula of the type:

$$mg ( Δ p , C g ) = V \cdot ( Φ - ω c ( Δ p , C g ) ) \cdot \frac{M g}{1000} ,$$

where V is the volume of said sample, Φ is the total porosity of said sample, Mg is the density of said adsorbable gas, ωc(Δp,Cg) is said kinematic porosity determined for said pressure gradient ΔP and said adsorbable gas concentration C_{g}.

Advantageously, an apparent permeability Kₐₚₚ can also be determined for said pressure gradient ΔP applied to said sample with a formula of the type:

$$K a p p = \frac{2 \text{?} L \text{?} Q \text{?} P 1}{S \text{?}} \text{?} 1013 , \\ \text{?} \text{indicates text missing or illegible when filed}$$

where Q is said flow rate (m³/s), μ is the viscosity of said inert gas (Pa·s), S is the section of said sample (m²), L is the length of said sample (m), P1 is the pressure applied upstream from the sample (Pa) and P2 is the pressure applied downstream from the sample (Pa).

According to a variant embodiment of the invention, an intrinsic permeability of said sample can further be determined by carrying out at least said following steps:
A. repeating step A for a plurality of pressure gradients and determining an apparent permeability value Kₐₚₚ for each of said pressure gradients of said plurality of gradients,
B. representing said values of said apparent permeabilities determined for each of said gradients as a function of an inverse of the average pressure Pm, said average pressure being defined by Pm=(P1+P2)/2,
C. determining said intrinsic permeability by determining the origin of a line passing through said values of said apparent permeabilities represented as a function of said inverse of said average pressure.

According to another variant embodiment of the invention, steps A, B and C can be applied for first and second pressure gradients, a first and a second kinematic porosity can be determined, and an adsorption variation induced by a variation of said pressure gradient can be characterized from the difference between said first and second kinematic porosities.

Alternatively, steps A, B and C can be applied for first and second adsorbable gas concentrations, a first and a second kinematic porosity can be determined, and an adsorption variation induced by a variation of said adsorbable gas concentration can be characterized from the difference between said first and second kinematic porosities.

Advantageously, said sample can be a rock sample from a petroleum reservoir and said pressure gradient for applying steps A and B can be close to the pressure in said reservoir.

According to this implementation of the invention, a development scheme can further be determined for said petroleum reservoir using a flow simulator, said kinematic porosity being at least one of the input parameters of said flow simulator.

### BRIEF DESCRIPTION OF THE FIGURES

Other features and advantages of the method according to the invention will be clear from reading the description hereafter of embodiments given by way of non-limitative example, with reference to the accompanying figures wherein:

FIG. 1 shows a breakthrough curve measured downstream from a porous medium sample,

FIG. 2 illustrates an example of a device suited for implementing the method according to the invention, and

FIG. 3 shows an example of an apparent permeability measurement as a function of the inverse of the average pressure applied to a porous medium sample.

### DETAILED DESCRIPTION OF THE INVENTION

In general terms, one object of the invention relates to a method of determining a quantity relative to the adsorption of an adsorbable gas in a sample of a porous medium, by determining a kinematic porosity of the sample considered, the kinematic porosity being determined for a given pressure gradient and adsorbable gas concentration.

What is referred to as “porosity” or “intrinsic porosity”, or “total porosity”, is the ratio of the void volume in the porous medium sample to the total volume of the sample. The total porosity thus corresponds to the volume of all the pores of a porous medium, whether connected or isolated. The term “total porosity” is used hereafter.

The “effective porosity” is understood to be a pore volume “useful” to the flow. In a water-saturated medium, it is defined as the volume of water that is extracted by gravity to the total volume. The effective porosity is thus obtained by subtracting the volume of bound water (water attached by capillarity to the wall of pores) and the volume of unconnected pores from the total porosity.

The present invention is based on the estimation of a porosity known as “kinematic”. The kinematic porosity is close to the effective porosity, and the two terms are often used indiscriminately in hydrology. The kinematic porosity however has a precise definition, which is the one used in the present invention: it corresponds to the ratio of the Darcy velocity (calculated according to Darcy's law over a section S) to the real velocity of flow of the water (through the cross-section, i.e. a fraction of surface S). The kinematic porosity co, evolves as a function of the affinity of the fluid with the porous medium (adsorption phenomenon as a function of the pressure applied). Thus, for example, in case of adsorption of chemical elements on the surface of the pores of the porous medium considered, the pore volume decreases, and therefore the kinematic porosity decreases. In other words, the porosity available to the fluid flow is reduced by the quantity of chemical elements adsorbed on the surface of the pores.

The present invention extends the concept of kinematic porosity, defined and known in hydrology for a fluid of liquid type, to a fluid of gaseous type.

The porous medium according to the invention can be any porous medium where at least one gaseous fluid can circulate and/or adsorb, by way of non-limitative example a rock of an underground formation, a filter or concrete. The present invention is in particular suited and/or advantageous to be applied in the case of a porous medium of very low permeability, such as a petroleum reservoir of very low permeability or tight gas reservoir.

The method according to the invention is implemented from a sample of the porous medium of interest. In the case of a porous medium of underground formation rock type, the core sample can be taken for example by drilling through the underground formation of interest, or it can originate from cuttings resulting from drilling operations through the formation of interest.

Prior to implementing the invention, the dimensions of the porous medium sample considered are measured, such as the diameter d (in m) and the length L (in m) of the sample, and the area S of the sample section (in m²) is deduced therefrom according to a formula of the type:

$$S = \text{?} {( \frac{d }{2} )}^{2}$$

$$\text{?} \text{indicates text missing or illegible when filed}$$

as well as the volume V (in m³) of the sample, with a formula of the type:

V=S×L.

The gas whose adsorption is to be quantified can be a hydrocarbon compound such as methane for example, or CO₂, or any other gas adsorbable on the surface of the pores of a porous medium.

The method according to the invention comprises at least three main steps described hereafter.

### 1. Darcy Velocity Measurement

The Darcy velocity associated with the sample of the porous medium considered is measured in this step.

The following substeps are therefore carried out:
injecting an inert gas such as helium or argon into a sample by applying a pressure gradient ΔP between upstream and downstream of the sample, and keeping this gradient constant. More precisely, a gradient ΔP=P1−P2 is applied, where P1 corresponds to the pressure applied upstream, P2 corresponds to the pressure applied downstream, and P1 must be greater than P2,
measuring the flow Q (in m³/s) generated by this pressure gradient between upstream and downstream of the sample kept constant. According to an implementation of the invention, this flow rate is measured at the sample outlet using a flowmeter,
determining the Darcy velocity V_{d} (in m/s), also referred to as fictitious Darcy velocity, which is a function of pressure gradient ΔP, using a formula of the type:

$${V}_{d} ( Δ P ) = \frac{Q}{S} .$$

According to an implementation of the invention, a pressure regulator is used to establish the pressure gradient to which the porous medium sample considered is subjected. Advantageously, pressure P2 downstream from the sample is the atmospheric pressure.

According to an implementation of the invention, the apparent permeability Kₐₚₚ (in m²) of the porous medium sample considered can also be determined with a formula of the type:

$$K a p p = \frac{2 \text{?} μ \text{?} L \text{?} Q \text{?} P 1}{S \text{?} ( P {1}^{2} - P {2}^{2} )} \text{?} 1013 , \\ \text{?} \text{indicates text missing or illegible when filed}$$

with:
Q: apparent flow (m³/s)
μ: inert gas viscosity (Pa·s)
S: sample section (m²)
L: porous medium length (shale sample) (m)
P1: pressure upstream from the sample (Pa)
P2: pressure downstream from the sample (Pa).

### 2. Measurement of the Adsorbable Gas Breakthrough Velocity

This step consists in measuring the breakthrough velocity of the adsorbable gas of interest, i.e. the velocity at which the adsorbable gas of interest “flows through” the porous medium sample. In general, this breakthrough velocity is a function of the pressure gradient to which the sample is subjected and of the concentration of the adsorbable gas injected into the sample.

According to the invention, the breakthrough velocity of the adsorbable gas is determined from the measurement of a curve referred to as breakthrough curve of the adsorbable gas of interest. Conventionally, for this type of experiment, the adsorbable gas used is referred to as tracer gas, and the breakthrough velocity, denoted by Vₜ, is also referred to as tracer gas velocity. Furthermore, the concentration in injected tracer gas or, in other words, the concentration in adsorbable gas of interest is denoted by C_{g} hereafter.

According to the invention, step 2 is carried out by applying the same pressure gradient ΔP between upstream and downstream of the sample as in step 1. According to the invention, step 2 is applied to the inert gas-saturated sample. According to an advantageous implementation of the invention, pressure gradient ΔP of step 1 is kept constant for implementing step 2.

Then, at a time t, an adsorbable gas (such as methane or any other adsorbable gas) is injected into the sample considered, upstream from the sample. The quantity of adsorbable gas that has flowed through the sample is then measured downstream from the porous sample, as a function of time or, in other words, for a plurality of times later than time t. Conventionally, the “breakthrough curve” is understood to be the curve representative of the evolution of the quantity of adsorbable gas that has flowed through the sample as a function of time. In general, due to its shape, this curve provides information about the retention of the adsorbable gas in the porous medium.

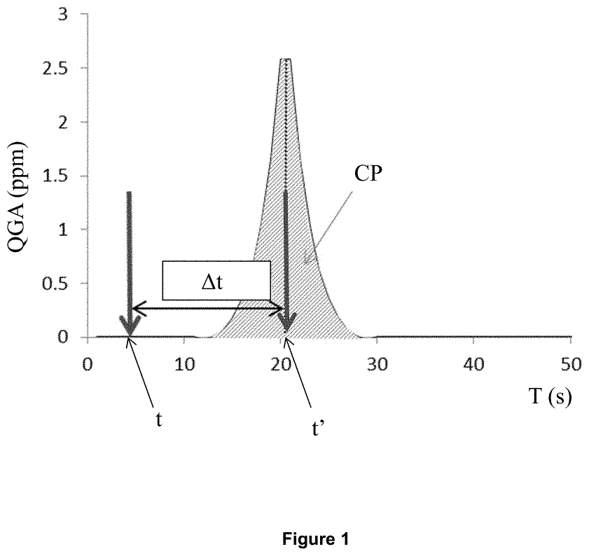

More preferably, the volume of adsorbable gas injected for this step is less than the volume of the pores of the porous medium sample considered. This injection precaution allows to obtain a Gaussian type breakthrough curve. An example of such a Gaussian type curve is given in FIG. 1 by way of illustration. This figure illustrates a breakthrough curve CP representative of the evolution of the adsorbable gas quantity QGA measured downstream from the sample as a function of time T. When the volume of gas injected is not less than the volume of the sample pores, the breakthrough curve can exhibit a maximum in form of a plateau instead of a well-differentiated peak, this plateau shape being related to a steady flow generated by the injection of a large volume of gas.

According to an implementation of the invention, the quantity of adsorbable gas that has flowed through the sample as a function of time can be measured using a low-concentration gas detector, such as a gas chromatograph or a spectrophotometer.

According to the invention, from the measurements of the adsorbable gas quantity that has flowed through the sample as a function of time thus obtained, a time difference At is determined between the time t of injection of the adsorbable tracer gas upstream from the sample and the time t′ of the maximum of the breakthrough curve (corresponding to the maximum of the adsorbable gas quantity measured downstream from the sample for the plurality of times). A graphical determination of this time difference Δt=t′−t is illustrated in FIG. 1 described above. The maximum of the breakthrough curve (Gaussian peak for this example) is thus graphically determined and the abscissa t′ of this maximum is deduced therefrom. Additionally or alternatively, instant t′ of the breakthrough curve maximum can be determined numerically, by seeking a maximum of values among the measurements, or by seeking the maximum of an analytical function representative of said measurements.

Then, according to the invention, the breakthrough velocity of the tracer gas Vₜ is determined, which is a function of pressure gradient ΔP to which the sample was subjected and of the injected adsorbable gas concentration C_{g}, with the formula as follows:

Vₜ(ΔP,C_{g})=Δt/L.

According to an implementation of the invention for which the volume and/or the mass of adsorbable gas injected into the sample in step 2 is known, it is also possible to determine the mass of tracer gas at the sample outlet from the surface of the breakthrough curve measured as described above. From the difference between the mass of tracer gas injected into the sample and the mass of tracer gas at the sample outlet (or, in other words, by mass balance), the mass of gas adsorbed in the porous matrix of the rock sample is determined for the pressure gradient ΔP applied and for the concentration C_{g} of the adsorbable gas injected.

### 3. Kinematic Porosity Determination

At the end of steps 1 and 2 described above, we respectively obtain a Darcy velocity V_{d} related to a flow of inert gas particles at a given pressure gradient ΔP and the velocity of an injected tracer gas considered Vₜ, which is a function of pressure gradient ΔP to which the sample was subjected and of the injected adsorbable gas concentration C_{g}.

According to the invention, in this step, a kinematic porosity ω_{c} is determined (in %) from the ratio of these two velocities, using the formula:

$$w c ( Δ P , {C}_{g} ) = \frac{{V}_{d} ( Δ P )}{{V}_{t} ( Δ P , {C}_{g} )} .$$

The kinematic porosity is in fact a function of pressure gradient ΔP to which the porous medium sample was subjected and of the tracer gas concentration C_{g}.

According to the invention, a quantity relative to the adsorption of an adsorbable gas in a porous medium sample is thus determined.

The quantity relative to the adsorption of gas in the porous medium of interest can be directly the kinematic porosity determined above. According to this implementation of the invention, the adsorption of the adsorbable gas that has been injected into the porous medium sample is characterized for the pressure gradient ΔP to which the sample was subjected and for the injected adsorbable gas concentration C_{g}, from the kinematic porosity itself. Indeed, the kinematic porosity provides information on the porosity really available to the flow, the kinematic porosity being reduced in relation to the total porosity (i.e. the pore volume, without fluid) of the sample due to the chemical elements adsorbed on the surface of the pores of the porous medium. The kinematic porosity is therefore itself a quantity relative to the adsorption of an adsorbable gas in a porous medium sample.

According to another implementation of the invention, a quantity relative to the adsorption of an adsorbable gas in a porous medium sample can be determined by determining a volume of gas and/or a mass of gas adsorbed in this porous medium sample, as a function of the kinematic porosity determined for the pressure gradient ΔP to which the sample was subjected and for the injected adsorbable gas concentration C_{g}.

According to an implementation of the invention, it is possible to deduce, from the volume V (m³) of the porous medium sample studied and from the total porosity Φ of this sample, a volume of gas V_{g} adsorbed in the porous medium sample, by means of a formula of the type:

V_{g}(Δp,Cg)=V·(Φ=ωc(Δp,Cg)), in m³,

The specialist has perfect knowledge of techniques for measuring the total porosity Φ (%) of a porous medium sample. A mercury or helium porosimetry technique or the NMR (nuclear magnetic resonance) technique can for example be implemented.

Alternatively or cumulatively, the adsorption of a given adsorbable gas in a porous medium is characterized by determining the adsorbed gas mass m_{g} from the kinematic porosity determined for the pressure gradient ΔP to which the sample was subjected and for the injected adsorbable gas concentration C_{g}. The adsorbed gas mass m_{g} can notably be determined with a formula of the type:

$$mg ( Δ p , C g ) = V \cdot ( Φ - ω c ( Δ p , C g ) ) \cdot \frac{M g}{1000} , in g .$$

where V (m³) is the volume of the porous medium sample studied, Φ the total porosity of this sample and Mg the density of the adsorbable gas.

Thus, the method according to the invention allows to determine a quantity relative to the adsorption of an adsorbable gas in a porous medium sample, from the porosity restriction induced by the adsorbed gas, for a pressure gradient ΔP applied to the sample considered and a gas concentration C_{g}.

According to an implementation of the invention where the porous medium sample is a rock sample from a petroleum reservoir, a pressure gradient close to the pressure in the reservoir studied is advantageously used for applying steps 1 and 2 of the method according to the invention.

In particular, the kinematic porosity determined in step 3 of the method according to the invention, for a pressure gradient close to in-situ conditions, can be used to determine a development scheme for the petroleum reservoir. For example, determination of a development scheme for a hydrocarbon reservoir comprises defining a number, a geometry and a site (position and spacing) for injection and production wells, determining an enhanced recovery type (waterflooding, surfactant flooding, etc.), etc. A hydrocarbon reservoir development scheme should for example enable a high rate of recovery of the hydrocarbons trapped in the geological reservoir identified, over a long operational life, requiring a limited number of wells and/or infrastructures. It is obvious that knowledge of the effective porosity really useful to the flow of gas present in the reservoir considered is an important parameter for determining such a development scheme. In particular, such data can be used as input parameters for a reservoir simulator such as the Puma Flow® software (IFP Energies nouvelles, France).

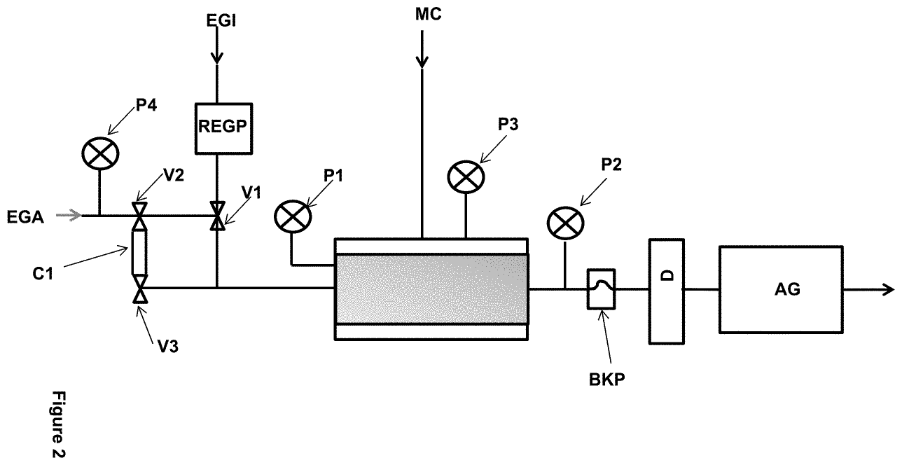

According to an embodiment of the invention, by way of non-limitative example, the device shown in FIG. 2 can be used for implementing the invention, this example of a device comprising the following elements:
C1: vessel suited to contain an adsorbable gas volume
P1: pressure detector for measuring the upstream pressure
P2: pressure detector for measuring the downstream pressure
P3: pressure detector for measuring the confining pressure
P4: pressure detector for measuring the pressure of vessel C1
V1: 3-way valve allowing bypass of the adsorbable gas flow
V2: 3-way valve for injection of the adsorbable gas
V3: solenoid valve
BKP: downstream pressure regulator
REGP: upstream pressure regulator
D: flowmeter for measuring the gas flow at the sample outlet
AG: low-concentration gas analyzer, such as a gas chromatograph or a spectrophotometer
EGA: adsorbable gas inlet
EGI: inert gas inlet
C: sample holder containment cell
MC: means for inducing a confining pressure.

According to this variant embodiment of the invention, steps 1 and 2 of the method according to the invention are carried out as follows:

### Step 1

In order to measure the Darcy velocity, the sample (grey shaded in FIG. 2) is placed in sample holder cell C and an isotropic confinement is applied around the sample. Pressure detector P3 records the confining pressure.

Valve V1 is open from inert gas inlet EGI to the sample and it is closed towards valve V2. The inert gas is kept constant at pressure P1 by means of pressure regulator P1. Valves V2 and V3 are closed. Pressure P1 is 20 bar for example.

Downstream from the sample, the downstream pressure is at atmospheric pressure and pressure detector P2 records the pressure downstream from the sample.

Flowmeter D measures the gas flow at the outlet generated by the pressure gradient between upstream pressure P1 and downstream pressure P2.

Gas analyzer AG identifies the gases at the outlet.

### Step 2

In order to measure the breakthrough velocity of the adsorbable gas, vessel C1 is filled with an adsorbable gas or tracer gas via valve V2 open onto adsorbable gas inlet EGA. Valve V3 is closed. The rest of the experimental setup remains in the configuration of step 1 at first.

Pressure detector P4 records the pressure applied in vessel C1. P4 must be equal to P1.

Once P4 and P1 equal, at a time t, valve V1 is opened onto V2, valve V2 is opened onto solenoid valve V3 and solenoid valve V3 is opened.

Downstream from the sample, the downstream pressure is at atmospheric pressure and pressure detector P2 records the pressure downstream from the sample.

Flowmeter D measures the gas flow at the outlet generated by the pressure gradient between upstream pressure P1 and downstream pressure P2.

Gas analyzer AG identifies and quantifies the gases at the outlet, and it notably records as a function of time the time-dependent breakthrough of the adsorbable gas.

### Variant 1: Determination of an Adsorption Variation

According to a first variant of the invention, an adsorption variation of the porous medium is determined as a function of a variation of the pressure gradient applied to the porous sample considered or of the adsorbable gas concentration.

According to an implementation of the first variant of the invention, steps 1 to 3 described above are repeated for a second pressure gradient ΔP₂, but for the same gas concentration C_{g}, and a kinematic porosity value ω_{c2} is determined for this second pressure gradient ΔP₂, in addition to the kinematic porosity ω_{c1} determined for first pressure gradient ΔP₁.

The adsorption variation induced by a pressure gradient variation can then be quantified by the difference between the two kinematic porosities. This adsorption variation as a function of a pressure gradient variation can further be characterized by estimating the variation of the adsorbed gas mass and/or of the adsorbed gas volume as a function of the pressure gradient.

Thus, according to an implementation of this variant of the invention, a relative adsorbed gas volume V_{gr} can be determined from the difference between these two kinematic porosities determined for these two pressure gradients, i.e.:

V_{gr}(ΔP₁,ΔP₂)=V·|w_{g2}−w_{g1}|, for Cg=const.

A relative adsorbed gas mass m_{gr} can be alternatively or cumulatively determined between these two measurement conditions ΔP₁ and ΔP₂, with the formula:

m_{gr}(ΔP₁,ΔP₂)=|m_{g}(ΔP₁)−m_{g}(ΔP₂)|for Cg=const.

According to another implementation of the first variant of the invention, steps 1 to 3 described above are repeated for a second concentration C_{g2} but for the same pressure gradient ΔP, and a kinematic porosity value ω_{c2} is determined for this second concentration C_{g2}, in addition to the kinematic porosity ω_{c1} determined for first concentration C_{g1}.

The adsorption variation induced by an adsorbable gas concentration variation can then be quantified by the difference between the two kinematic porosities. This adsorption variation as a function of the adsorbable gas concentration can further be characterized by estimating the variation of the adsorbed gas mass and/or of the adsorbed gas volume as a function of the adsorbable gas concentration.

According to an implementation of this variant of the invention, a relative adsorbed gas volume can be determined from the difference between these two kinematic porosities determined for these two gas concentrations, i.e.:

V_{gr}(C_{g1},C_{g2})=V·|w_{g2}−w_{g1}|, for ΔP=const.

A relative adsorbed gas mass can be alternatively or cumulatively determined between these two measurement conditions C_{g1} and C_{g2}, with the formula:

m_{gr}(C_{g1},C_{g2})=|m_{g}(C_{g1})−m_{g}(C_{g2})|, for ΔP=const.

### Variant 2: Determination of the Kinematic Porosity Evolution as a Function of the Pressure Gradient and/or of the Adsorbable Gas Concentration

According to an embodiment of the invention, steps 1 to 3 described above are repeated for a plurality of pressure gradients ΔPₙ, and a curve representative of the kinematic porosity evolution as a function of the pressure gradient applied to the sample is determined. It is then possible to predict adsorbed gas quantities (mass and/or volume for example) as a function of the pressures applied to the sample. In particular, the higher the pressure gradient applied, the lower the kinematic porosity.

According to an embodiment of the invention that can be carried out alternatively to or cumulatively with the embodiment described above, steps 1 to 3 described above can be repeated for a plurality of concentrations C_{gn}, and a curve representative of the kinematic porosity evolution as a function of the injected adsorbable gas concentration is determined. It is then possible to predict adsorbed gas quantities (mass and/or volume for example) as a function of the injected gas concentration. In particular, the higher the injected adsorbable gas concentration, the lower the kinematic porosity.

Thus, in general terms, when the kinematic porosity determination is thus repeated for a plurality of pressures and/or gas concentrations, the specialist can deduce therefrom a gas adsorption and/or desorption capacity for the porous medium sample considered.

Notably, from such kinematic porosity curves plotted as a function of the pressure and/or the gas concentration, the specialist can for example deduce optimal operability conditions for the porous medium of interest. For example, when the porous medium sample considered comes from an underground formation whose gaseous hydrocarbons are to be exploited, the specialist then is provided with information that will allow him to best plan the development of this gas deposit. Indeed, the specialist thus knows the evolution of the effective porosity really useful to the gas flow in the reservoir considered, which provides information on the gas adsorption/desorption capacity of the rock making up this reservoir. Such data contribute to an assessment of the gas production potential of the reservoir.

This information can also allow to plan a development scheme for this reservoir. For example, determination of a development scheme for a hydrocarbon reservoir comprises defining a number, a geometry and a site (position and spacing) for injection and production wells, determining an enhanced recovery type (waterflooding, surfactant flooding, etc.), etc. A hydrocarbon reservoir development scheme should for example enable a high rate of recovery of the hydrocarbons trapped in the geological reservoir identified, over a long operational life, requiring a limited number of wells and/or infrastructures. It is obvious that knowledge of the effective porosity really useful to the flow of gas present in the reservoir considered is an important parameter for determining such a development scheme. In particular, such data can be used as input parameters for a reservoir simulator such as the Puma Flow® software (IFP Energies nouvelles, France).

Then, once a development scheme defined, the hydrocarbons trapped in the reservoir are exploited according to this development scheme, notably by drilling the injection and production wells of the development scheme thus determined, and by installing the production infrastructures necessary for the development of the reservoir.

### Variant 3: Intrinsic Permeability Measurement

According to a third variant of the invention, the intrinsic permeability of the sample is also determined. To correct the measurement bias related to the gas particle slippage during a gas permeability measurement, a correction that is function of the Klinkenberg coefficient is applied.

To determine the Klinkenberg coefficient, step 1 described above is repeated for at least two other pressure gradients or, in other words, step 1 described above is applied for at least three pressure gradients.

Then, for each pressure gradient, an apparent permeability Kₐₚₚ (in m²) is determined with a formula of the type:

$$K a p p = \frac{2 \text{?} μ \text{?} L \text{?} Q \text{?} P 1}{S \text{?} ( P {1}^{2} - P {2}^{2} )} \text{?} 1013 , \\ \text{?} \text{indicates text missing or illegible when filed}$$

with:
Q: apparent flow (m³/s)
P: inert gas viscosity (Pa·s)
S: sample section (m²)
L: porous medium length (shale sample) (m)
P1 pressure upstream from the sample (Pa)
P2 pressure downstream from the sample (Pa).

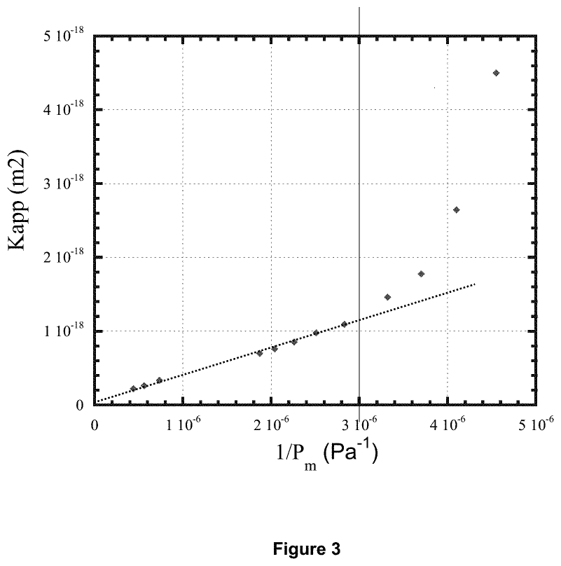

It is possible for example to graphically represent these apparent permeability measurements Kₐₚₚ as a function of the inverse of the average pressure denoted by 1/Pm, with Pm=(P1+P2)/2. FIG. 3 illustrates an example of such a curve. It is observed that the points (Kₐₚₚ; Pm) obtained with the highest pressure gradients align on a line referred to as Klinkenberg line. This line can also be written as follows:

Kapp=K∞+(β·K∞)·1/Pm

with:
k∞: the intrinsic permeability of the sample (m²)
β·K∞: slope of the Klinkenberg line.

Thus, from this line, the intrinsic permeability k∞ is determined, which is defined as the origin of the Klinkenberg line.

According to an implementation of the invention, the origin of the Klinkenberg line can be graphically determined. Alternatively, the origin of the Klinkenberg line can also be determined by means of a linear regression.

### Embodiment Example

The features and advantages of the method according to the invention will be clear from reading the application example hereafter.

The method according to the invention is applied to a clay type rock sample from the Vaca Muerta formation in Argentina. The sample has a diameter d of 40 mm and a length L of 27 mm. The inert gas according to the invention is helium and the adsorbable gas according to the invention is methane. The helium porosity or total porosity previously measured for this sample is 6%.

Steps 1 to 3 of the method according to the invention are first applied for a gradient ΔP₁ of 50 bar.

Step 1 is applied according to this pressure gradient, a gas flow Q is measured downstream from the sample using a flowmeter (at ΔP₁ constant and stabilized at 50 bar) and a Darcy velocity is determined, vd=1.29×10⁻⁷ m/s, as described in step 1.

Step 2 of the method according to the invention is applied for the same pressure gradient as step 1, i.e. ΔP₁=50 bar. A volume of 20 cm³ methane at 2000 ppm is injected at 50 bar into the upstream circuit of the experimental setup. The 50 bar pressure is equivalent to the upstream pressure P1 already applied in step 1. There is therefore no change in the pressure gradient between steps 1 and 2. The methane concentration on the downstream side of the sample is measured over time. A curve showing the methane breakthrough through the sample as a function of time is obtained. The breakthrough velocity of the tracer (methane) is determined as described in step 2 above, i.e. vt=2.17×10⁻⁶ m/s.

At the end of step 3, the kinematic porosity ω_{c1} is determined for a pressure gradient ΔP₁=50 bar by calculating the ratio between the Darcy velocity and the tracer velocity. A kinematic porosity ω_{c1} of 5.9% is obtained for pressure gradient ΔP₁.

The procedure is repeated for a pressure gradient ΔP₂=100 bar for the application of steps 1 and 2. The volume of methane in step 2 remains 20 cm³ at 2000 ppm, but this time it is injected at 100 bar.

The Darcy velocity and the tracer velocity are deduced therefrom for this new gradient ΔP₂. A Darcy velocity Vd at 100 bar equal to 1.27×10⁻⁷ m/s and an adsorbable gas breakthrough velocity Vt at 100 bar equal to 2.26×10⁻⁶ m/s are obtained.

A second kinematic porosity ω_{c2}=5.6% is deduced.

As described above in variant 1 of the method according to the invention, a 0.2 μg adsorbed gas mass difference is deduced between a gradient of 50 bar and a gradient of 100 bar.

Besides, the intrinsic permeability of the sample is also determined as described in variant 3 above. An intrinsic permeability of 214 mD is thus determined.

This measurement is in accordance with prior results published in the document:
Romero-Sarmiento, Maria-Fernanda, et al., «Geochemical and petrophysical source rock characterization of the Vaca Muerta Formation, Argentina : Implications for unconventional petroleum resource estimations», International Journal of Coal Geology 184 (2017) : 27-41.

Thus, the method according to the invention allows, from conventional experimental measurements, fast and easy to implement, to reliably determine a quantity of gas adsorbed in a sample of a porous medium.

Furthermore, when the kinematic porosity determination is repeated for a plurality of pressures and/or gas concentrations, a gas adsorption capacity of the porous sample considered can be deduced, as a function of the pressures and/or concentrations applied. Advantageously, confining pressures and/or gas concentrations close to in-situ exploitation conditions are used.

1. A method of determining at least one quantity relative to the adsorption of at least one adsorbable gas in a sample of a porous medium, wherein at least the following steps are carried out:
a) applying a pressure gradient between upstream and downstream of the sample and injecting an inert gas upstream from the sample subjected to the pressure gradient;
measuring at least one flow rate of the inert gas downstream from the sample, and determining a Darcy velocity from the flow rate of the measured inert gas,
b) for the pressure gradient applied between upstream and downstream of the sample, the sample being saturated with the inert gas, injecting an adsorbable gas upstream from the sample at a first time t, the adsorbable gas having a concentration C_{g}; downstream from the sample and for a plurality of times later than the first time, measuring a quantity of the adsorbable gas that has passed through the sample;
determining a breakthrough velocity for the adsorbable gas from the time t′ of a maximum of the curve representative of the time-dependent evolution of the measured adsorbable gas quantity for the plurality of times,
c) determining a kinematic porosity as a function of the pressure gradient applied to the sample and of the concentration in the adsorbable gas from the ratio of the Darcy velocity to the breakthrough velocity of the adsorbable gas, and determining, for the pressure gradient applied to the sample and for the concentration in the adsorbable gas, a quantity relative to the adsorption of the adsorbable gas in the sample from the kinematic porosity.

2. A method as claimed in claim 1 wherein, in step B, the volume of the injected adsorbable gas is less than the volume of the pores of the sample.

3. A method as claimed in claim 1, wherein the breakthrough velocity Vₜ of the adsorbable gas is determined with a formula of the type: Vₜ(ΔP,C_{g})=Δt/L, where Δt=t′−t, L is the length of the sample, ΔP is the pressure gradient and C_{g} is the concentration in the adsorbable gas.

4. A method as claimed in claim 1, wherein the quantity relative to the adsorption is a volume of gas adsorbed in the sample and/or a mass of gas adsorbed in the sample.

5. A method as claimed in claim 4, wherein the adsorbed gas volume Vg in the sample is determined with a formula of the type:
Vg(Δp,Cg)=V·(Φ−ωc(Δp,Cg)), in m³,
where V is the volume of the sample, Φ is the total porosity of the sample and ωc(Δp,Cg) is the kinematic porosity determined for the pressure gradient ΔP and the adsorbable gas concentration C_{g}.

6. A method as claimed in claim 4, wherein the adsorbed gas mass m_{g} in the sample is determined with a formula of the type:
$$mg ( Δ p , C g ) = V \cdot ( Φ - ω c ( Δ p , C g ) ) \cdot \frac{M g}{1000} ,$$
where V is the volume of the sample, Φ is the total porosity of the sample, Mg is the density of the adsorbable gas, ωc(Δp,Cg) is the kinematic porosity determined for the pressure gradient ΔP and the adsorbable gas concentration C_{g}.

7. A method as claimed in claim 1, wherein an apparent permeability Kₐₚₚ is also determined for the pressure gradient ΔP applied to the sample with a formula of the type:
$$K a p p = \frac{2 \text{?} μ \text{?} L \text{?} Q \text{?} P 1}{S \text{?} ( P 1 \text{?} - P 2 \text{?} )} \text{?} 1013 , \\ \text{?} \text{indicates text missing or illegible when filed}$$
where Q is the flow rate (m³/s), μ is the viscosity of the inert gas (Pa·s), S is the section of the sample (m²), L is the length of the sample (m), P1 is the pressure applied upstream from the sample (Pa) and P2 is the pressure applied downstream from the sample (Pa).

8. A method as claimed in claim 7, wherein an intrinsic permeability of the sample is further determined by carrying out at least the following steps:
A. repeating step A for a plurality of pressure gradients and determining an apparent permeability value Kₐₚₚ for each of the pressure gradients of the plurality of gradients,
B. representing the values of the apparent permeabilities determined for each of the gradients as a function of an inverse of the average pressure Pm, the average pressure being defined by Pm=(P1+P2)/2,
C. determining the intrinsic permeability by determining the origin of a line passing through the values of the apparent permeabilities represented as a function of the inverse of the average pressure.

9. A method as claimed in claim 1, wherein steps A, B and C are applied for first and second pressure gradients, a first and a second kinematic porosity are determined, and an adsorption variation induced by a variation of the pressure gradient is characterized from the difference between the first and second kinematic porosities.

10. A method as claimed in claim 1, wherein steps A, B and C are applied for first and second adsorbable gas concentrations, a first and a second kinematic porosity are determined, and an adsorption variation induced by a variation of the adsorbable gas concentration is characterized from the difference between the first and second kinematic porosities.

11. A method as claimed in claim 1, wherein the sample is a rock sample from a petroleum reservoir and the pressure gradient for applying steps A and B is close to the pressure in the reservoir.

12. A method as claimed in claim 11, wherein a development scheme is further determined for the petroleum reservoir using a flow simulator, the kinematic porosity being at least one of the input parameters of the flow simulator.
